(19) United States
(12) Patent Application Publication
(10) Pub. No.: US 2004/0210190 A1
(43) Pub. Date: Oct. 21, 2004

(54) Interventional diagnostic catheter and a method for using a catheter to access artificial cardiac shunts

(75) Inventors: Robert Kohler, Lake Elmo, MN (US); David Mowry, Waconia, MN (US); Timothy R. Conrad, Eden Prairie, MN (US)

Correspondence Address: FINNEGAN, HENDERSON, FARABOW, GARRETT & DUNNER, LLP, 1300 I STREET, NW, WASHINGTON, DC, 20005, US

(73) Assignee: Percardia, Inc.

(21) Appl. No.: 10/844,372

(22) Filed: May 13, 2004

Related U.S. Application Data
(63) Continuation of application No. 09/931,655, filed on Aug. 16, 2001.

(51) Int. Cl. A61B 6/00; A61M 31/00

(52) U.S. Cl. 604/93.01; 600/431

(57) ABSTRACT

One aspect of the present invention relates to a method for passing a fluid through a shunt located in the wall of a heart, the shunt providing fluid communication between a heart chamber and a coronary artery, with a hollow catheter. Another aspect of the present invention relates to a method of inserting a wire through a shunt located in the wall of a heart with a hollow catheter. A further aspect of the present invention relates to passing fluid through a shunt located in the wall of a heart, the shunt providing fluid communication between a heart chamber and a coronary artery, by injecting fluid into the heart chamber. A further aspect of the present invention relates to a catheter with a flexible, hollow, inner member to which a self expanding basket is attached. A further aspect of the present invention relates to a method of passing a radio-opaque contrast fluid through a shunt located in a heart wall, the shunt providing fluid communication between a heart chamber and a coronary artery. A further aspect of the present invention relates to inserting a wire into a coronary artery through a shunt located in a heart wall, the shunt providing fluid communication between a heart chamber and the coronary artery. A still further aspect of the present invention relates to a catheter including an inner tube with a self-expanding basket and an outer sheath about the inner tube. A further aspect of the present invention relates to a catheter with a flexible inner member with a shunt locating element at a distal end and an outer sheath about the inner member.

### FIELD OF THE INVENTION

The present invention relates to a method and apparatus for performing closed-chest cardiac diagnostic procedures and other cardiac intervention procedures using a catheter placed into the patient's heart. More specifically, this invention relates to accessing cardiac shunts which were previously placed in the heart wall for diagnostic and intervention purposes.

### BACKGROUND OF THE INVENTION

The placing of artificial shunts or other durable passageways in the heart wall to connect heart chambers containing oxygenated blood with coronary arteries is known. These devices and the techniques for placing them in the heart are described in detail in U.S. Pat. No. 5,944,019, issued Aug. 31, 1999, which is hereby incorporated by reference. Collectively, in this application, these devices, including artificial shunts and other durable passageways will be referred to solely as shunts. Such shunts typically are placed in the wall of the heart to allow oxygenated blood to flow into a partially or completely occluded coronary artery as an alternative to more traditional or conventional vein graft coronary arterial bypass procedures. What is needed are effective techniques for accessing the shunts for diagnostic reasons or other reasons.

### SUMMARY OF THE INVENTION

One aspect of the present invention relates to a method for passing a fluid through a shunt located in the wall of a heart, the shunt providing fluid communication between a heart chamber and a coronary artery, with a hollow catheter. Another aspect of the present invention relates to a method of inserting a wire through a shunt located in the wall of a heart with a hollow catheter. A further aspect of the present invention relates to passing fluid through a shunt located in the wall of a heart, the shunt providing fluid communication between a heart chamber and a coronary artery, by injecting fluid into the heart chamber. A further aspect of the present invention relates to a catheter with a flexible, hollow, inner member to which a self expanding basket is attached. A further aspect of the present invention relates to a method of passing a radio-opaque contrast fluid through a shunt located in a heart wall, the shunt providing fluid communication between a heart chamber and a coronary artery. A further aspect of the present invention relates to inserting a wire into a coronary artery through a shunt located in a heart wall, the shunt providing fluid communication between a heart chamber and the coronary artery. A still further aspect of the present invention relates to a catheter including an inner tube with a self-expanding basket and an outer sheath about the inner tube. A further aspect of the present invention relates to a catheter with a flexible inner member with a shunt locating element at a distal end and an outer sheath about the inner member.

### BRIEF DESCRIPTION OF THE DRAWINGS

The accompanying drawings, which are incorporated in and constitute a part of the description, illustrate several aspects of the invention and together with the description, serve to explain the principles of the invention. A brief description of the drawings is as follows:

FIG. 1 is a side view of an embodiment of an assembled catheter of the present invention.

FIG. 2 is a closer detail view the distal end of the assembled catheter of FIG. 1.

FIG. 3 is a side view of the outer sheath of the assembled catheter of FIG. 1.

FIG. 4 is an end view of the distal end of the outer sheath of FIG. 3.

FIG. 5 is a side view of the inner tube of the assembled catheter of FIG. 1.

FIG. 6 is a closer detail view of the distal end of the inner tube of FIG. 5.

FIG. 7 is a schematic illustration with a heart in partial cutaway of an embodiment of a catheter of the present invention to catheterize the left ventricle of a patient's heart via the femoral artery.

FIG. 8 is a close-up of the heart of FIG. 7, showing a distal end of the catheter within the patient's left ventricle and a shunt in place in the wall of the patient's heart.

FIG. 9 is a cross-sectional view of the heart wall with a shunt in place between the heart chamber and a coronary artery and the distal end of the catheter with the stabilizing collapsible basket attached to the inner tube collapsed and retracted within the outer sheath.

FIG. 10 is the cross-sectional view of FIG. 9 showing the distal end of the outer sheath of the catheter retracted to permit the basket to expand.

FIG. 11 is the cross-sectional view of FIG. 9, with the expanded basket now placed overlaying the protruding end of the shunt.

FIG. 12 is the cross-sectional view of FIG. 9 with the distal end of the outer sheath being extended toward the distal end of the inner tube causing the basket to collapse about the protruding end of the shunt and stabilize the catheter with respect to the shunt.

FIG. 13 is the cross-sectional view of FIG. 9, with the heavy arrows representing the flow of fluid being passed through the inner tube of the catheter and through the shunt, into the coronary artery.

FIG. 14 is the cross-sectional view of FIG. 9, showing a wire being inserted through the inner tube and through the shunt into the coronary artery.

FIG. 15 is a cross-sectional view of an alternative embodiment of a distal end of a catheter according to the present invention with the gripping element inverted within the inner tube.

FIG. 16 is a cross-sectional view of the catheter of FIG. 15 with a shaft inserted through the inner tube and forcing the gripping element from its inverted postion.

FIG. 17 is a cross-sectional view of the catheter of FIG. 16 with the shaft removed from the inner tube.

FIG. 18 is a cross-sectional view of the catheter of FIG. 17 with the gripping element retracted within the outer sheath.

FIG. 19 is an alternative embodiment of a distal end of a catheter according to the present invention for injecting dye into a heart chamber.

FIG. 20 is an alternative embodiment of a distal end of a catheter according to the present invention for injecting dye into a heart chamber.

FIG. 21 is an alternative embodiment of a distal end of a catheter according to the present invention for injecting dye into a heart chamber.

### DETAILED DESCRIPTION

With reference to the detailed drawing figures in which identical elements are numbered identically throughout, a description of the preferred embodiment and various alternative embodiments will now be provided.

Once a shunt has been placed in the heart wall as described in U.S. Pat. No. 5,944,019, there may arise the need to access the shunt for diagnostic or other reasons. For example, a physician may desire to inject radio-opaque chemical contrast material through the shunt to permit the use of cardiac imaging techniques to verify blood flow through the affected coronary artery downstream of the site of the shunt. Alternatively, it may be desirable to reach through the shunt to insert angioplasty tools to a site in the affected coronary artery downstream of the site of the shunt. Further, a physician may access the shunt to insert an arterial stent into the affected coronary artery at a site downstream from the shunt.

One of the least traumatic methods of accessing the heart and any shunts that might be implanted in the heart wall is with a catheter which enters the body via insertion through the femoral artery in the patient's groin and is advanced through the femoral artery, descending aorta and ascending aorta, into the heart. Catheters for femoral insertion are known. However, when accessing a shunt placed in the heart wall of a patient without cardiopulmonary bypass, actually inserting a tool or other device into the shunt and the artery downstream of the shunt can be quite difficult. Without cardiopulmonary bypass, the patient's heart must necessarily be contracting during the catheterization, making the environment around the shunt quite dynamic. Known catheterization methods and apparatus do not address this issue.

As a follow-up procedure to the placement of a shunt through the heart wall to a coronary artery, it may be desirable to explore blood flow in the artery downstream of the shunt to determine the efficacy of the shunt in bypassing an arterial occlusion. The most common method of determining blood flow within a coronary artery is to insert a catheter directly into the artery and introduce a radio-opaque chemical contrast. Then, using radiographic or other cardiac imaging techniques, the flow of blood within the artery can be seen. This method is effective in the traditional vein graft arterial bypass situation as a new arterial pathway is created and any occlusions in the artery are thus avoided. A catheter can be inserted into the artery directly via the aorta and contrast injected directly into the artery through the catheter. When a cardiac shunt is in place, this method is less feasible, since a new arterial path bypassing the occlusion most likely has not been created, meaning that injecting contrast into the artery via the aorta will be injecting contrast at a site above the occlusion which necessitated the bypass procedure. Rather, the shunt permits blood from a heart chamber with oxygenated blood to flow directly into the coronary artery at a site downstream of the occlusion. For cardiac imaging techniques to be effective in determining blood flow in the affected artery where a shunt has been placed, the contrast is preferably injected though the shunt into the artery so that flow downstream of the occlusion can be explored. With a cardiac shunt in place, the cardiac catheter is preferably inserted through the aorta into the heart chamber for contrast to be injected into the shunt and the artery downstream of the shunt. However, because the movement of blood creates currents and eddies within the heart chambers, merely injecting a contrast within the chamber where the shunt is located may not ensure that sufficient contrast will flow through the shunt and into the artery to permit the blood flow to be adequately imaged. Instead, the contrast is preferably injected directly into and through the shunt to permit effective imaging and flow analysis.

The present invention relates to a technique and devices for accessing shunts through heart walls. One aspect of the present invention relates to a technique and apparatus for allowing a catheter to enter the heart and align with or attach to an object imbedded in the heart wall with a high degree of certainty while a normal cardiac rhythm is maintained.

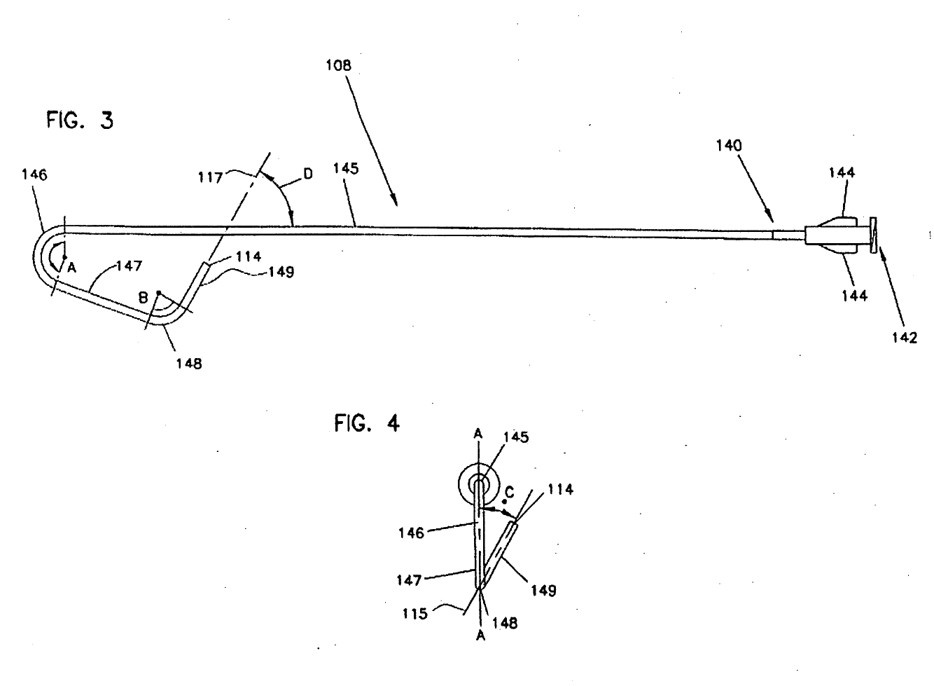

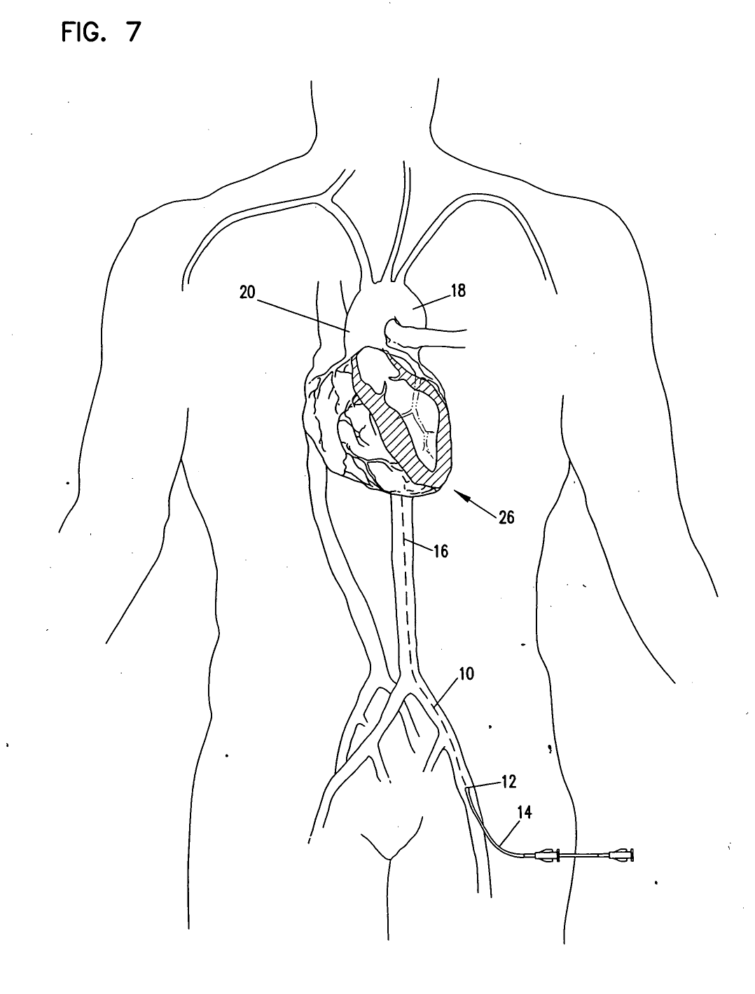

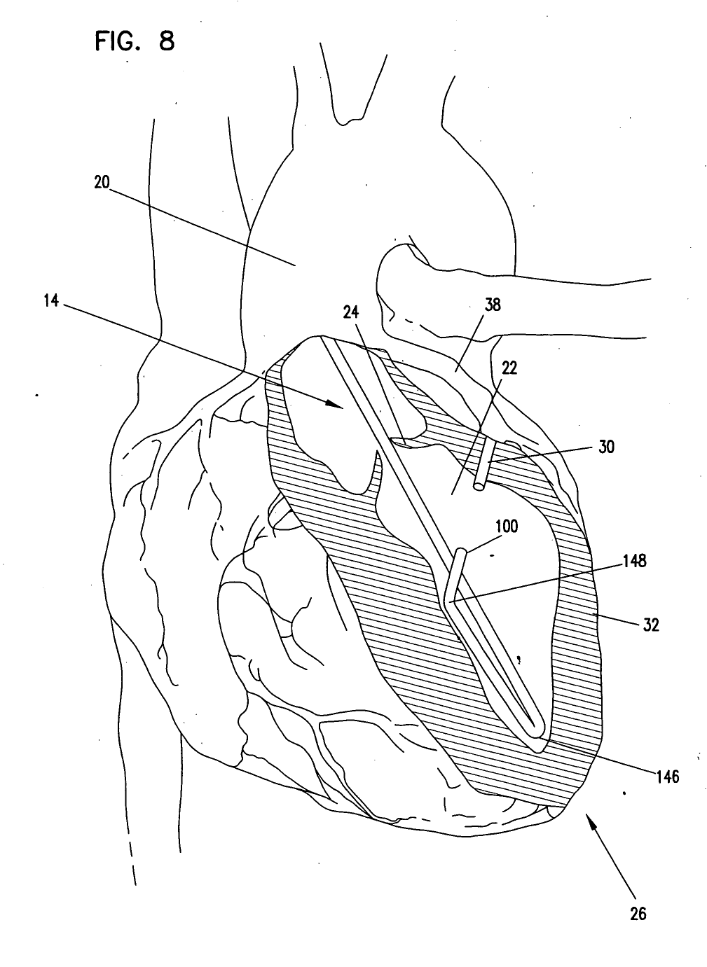

Now referring to FIGS. 1 through 6, an embodiment of a catheter apparatus 14 is shown. In FIGS. 3 and 4, outer sheath 108 of catheter 14 is shown in detail. At proximal end 140 of outer sheath 108, a hub 142 is attached. Hub 142 includes a pair of wings 144 extending radially from hub 142 to assist in the manipulation of the catheter and control the orientation of the curvature of catheter 14 when catheter 14 is inserted in a patient's body. Wings 144 extend on opposite sides of hub 142 and are oriented so as to be coplanar with primary curve 146 of catheter 14. Primary curve 146 and secondary curve 148 are designed to improve access to shunt 30 located in heart wall 32 within heart chamber 22. The relationship of primary curve 146 and secondary curve 148 of outer sheath 108, and the anatomic shape of the left ventricle, as well as the relationship of distal end 100 to shunt 30, are illustrated in FIGS. 7 and 8.

Primary curve 146 and secondary curve 148 combine to form a three-dimensional bend profile, as shown in FIGS. 3 and 4. Curves 146 and 148 separate outer sheath 108 and define three distinct segments. A first segment 145 extends from hub 142 to primary curve 146. First segment 145 is predominantly straight and preferably sized to extend from the femoral stick to the bottom of the left ventricle. A second segment 147 extends between primary curve 146 and secondary curve 148. A preferred length of the second segment is in the range of 1 to 9 centimeters. A third segment 149 is defined between secondary curve 148 and distal end 100. A preferred length of the third segment is in the range of 0.5 to 3 centimeters.

Outer sheath 108 is preferably made of a material that is flexible enough to allow catheter 14 to be straightened for insertion into and passage through the arterial path to the heart. At the same time, the material preferably has the elastic memory for returning to a pre-set shape, such as that shown in FIGS. 3 and 4. First segment 145, primary curve 146 and second segment 147 define a first plane AA. Primary curve 146 traverses an angle A in the range of one hundred forty to one hundred eighty degrees, preferably approximately one hundred and sixty degrees. In plane AA, secondary curve 148 traverses an angle B in the range of sixty to one hundred twenty degrees, most preferably approximately eighty degrees. Third segment 149 is inclined from plane AA by an angle C in the range of ten to fifty degrees, most preferably approximately 30 degrees. As shown in FIG. 4, third segment 149 is offset in a clockwise direction from second segment 147. In other embodiments, third segment 149 can be offset in a counter-clockwise direction from second segment 147. As shown in FIG. 4, third segment 149 is aligned along line 115 that does not intersect first segment 145. However, line 115 is preferably within a plane 117 (shown in FIG. 3) that intersects first segment 145 at an angle D in the range of sixty to one hundred forty degrees, most preferably approximately one hundred degrees. The preferred embodiment has outer sheath 108 made of medical grade thermoplastic elastomer resin. Other materials with similar qualities may be used for the outer sheath. It is also anticipated that the outer sheath will have no preset bends but may be capable of being formed into the above-described shape once the catheter has been inserted into the left ventricle.

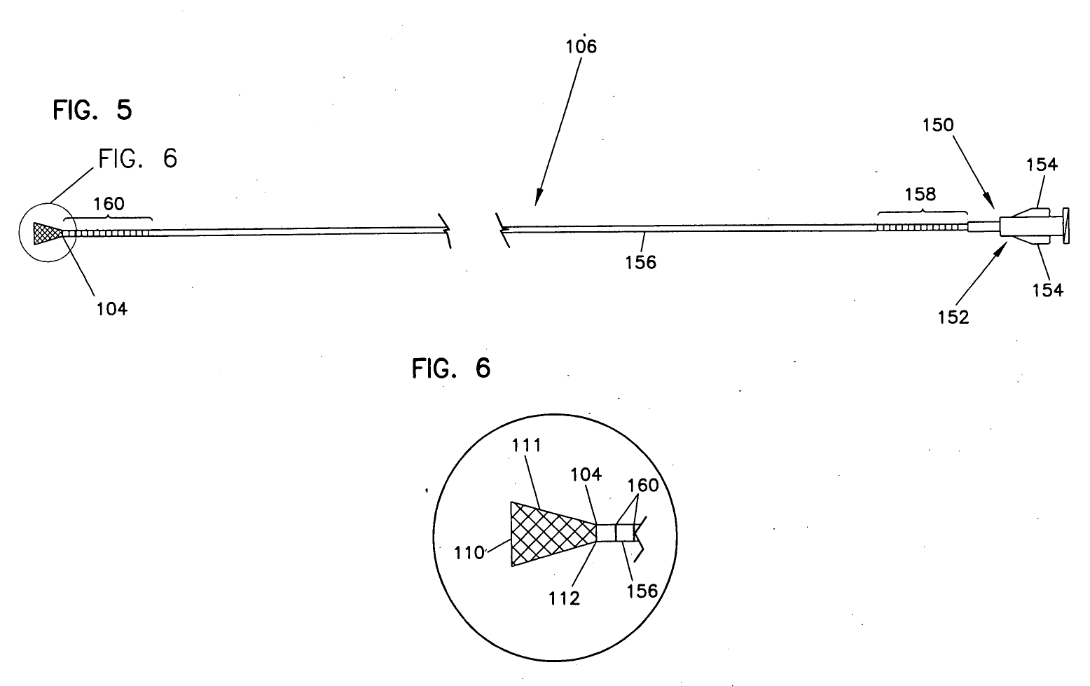

Referring now to FIGS. 5 and 6, inner tube 106 of catheter 14 is shown in detail. Inner catheter 106 includes a hub 152 at proximal end 150, a hollow catheter shaft 156 and distal end 104. At distal end 104 is attached expanding basket 102. Hub 152 includes a pair of wings 154 extending radially from hub 152 on opposite sides. Wings 152 permit the rotation and manipulation of inner tube 106 within outer sheath 108 and provide a reference for the user of catheter 14 as to the extent of movement and orientation of inner tube 106. Also at proximal end 150, beginning at hub 152, are a series of circumferential reference rings 158 about catheter shaft 156, spaced at one centimeter intervals for a distance of about 10 centimeters. Rings 158 aid the user in determining the relative extent of insertion of inner tube 106 within outer sheath 108. At distal end 104 of inner tube 106, a series of circumferential reference rings 160 are also placed about catheter shaft 156. Rings 160 include a radio-opaque material so that a fluoroscope or similar device can be used during the insertion and manipulation of catheter 14 to determine the location of distal end of inner tube 106 within the patient's body. Alternatively, or in addition to these reference rings, a fiber optical viewing system may be inserted within catheter shaft 156 with a viewing end located at the distal end of catheter 14 to provide visual imagery regarding the location of the distal end of catheter 14 and assist with its insertion and manipulation.

At distal end 104 of inner tube 106, a gripping element in the form of a self-expanding basket 102 is attached, as shown in FIG. 6. Basket 102 is shaped so that in a collapsed form 116 (shown in FIG. 12), it can be inserted within outer sheath 108 and completely contained within outer sheath 108. Provision may be made for permitting passage of objects such as an optical fiber viewing system through the distal end 104 of inner tube 106 to the distal end of catheter 14, while collapsed basket 116 is held within outer sheath 108. Basket 102 is preferably sized and shaped to allow overlay on first end 34 of shunt 30 in heart wall 32 (see FIG. 11). A frustal conical shape is shown in FIG. 6 and is the preferred embodiment but other shapes may also be suitable. Narrow end 112 of basket 102 is attached to distal end 104 of inner tube 106. Wide end 110 opens away from distal end 104 of inner tube 106 when basket 102 is allowed to expand. The preferred embodiment has basket 102 made of an elastic or super-elastic material such as nickel-titanium alloy. Other materials may be suitable for this application as well, as long as they have sufficient flexibility and resilience to permit being collapsed within outer sheath 108 and expending without additional influence when distal end 114 of outer sheath 108 is retracted. Alternatively, the gripping element may also be in the form of a lass-type snare.

Inner tube 106 is preferably made of a material with sufficient column strength to permit the axially movement of inner tube 106 within outer sheath 108 and controlled manipulation of distal end 104 and basket 102 attached thereto when catheter 14 is within the heart of a patient. The preferred embodiment has inner tube 106 made of a medical grade thermoplastic elastomer resin. Other plastic and metallic materials may be used provided they have the required physical characteristics. The material used to construct inner tube 106 preferably has a degree of lubricity with respect to the inner surface of outer catheter 108 to promote smoother relative movement of the two catheter components. If inner tube 106 material does not have a sufficient lubricity with respect to outer sheath 108 material, a low friction coating material can be applied to inner tube 106 prior to insertion into outer sheath 108.

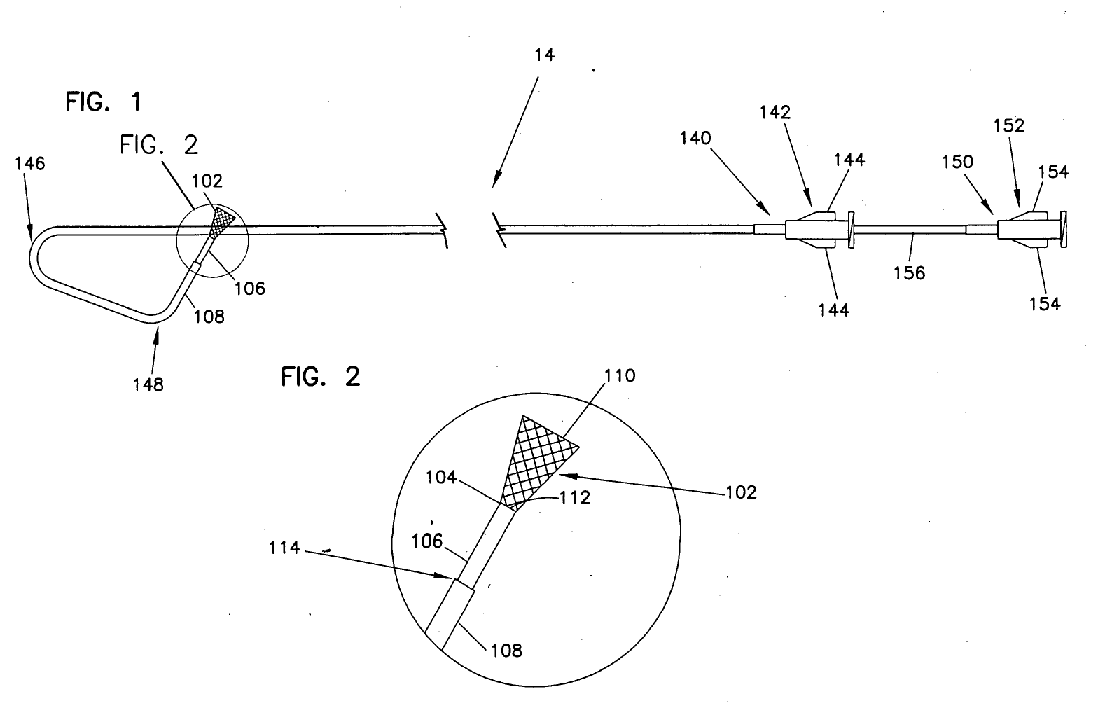

Assembled catheter 14 including inner tube 106, outer sheath 108 and basket 102 is shown in FIGS. 1 and 2. Inner tube 106 is axially slidably contained within outer sheath 108. In FIGS. 1 and 2, assembled catheter 14 is shown with basket 102 extended from distal end 114 of outer sheath 108 and in a fully expanded shape. The relationship of hub 152 of inner tube 106 and hub 142 of outer sheath 108 is shown by way of an example. Other relative orientations of the hubs are possible as long as the user is provided with a consistent reference as to the relationship of distal end 104 of inner tube 106 and distal end 114 of outer sheath 108.

Referring now to FIGS. 7 through 14, the use of one embodiment of the catheter of the present invention to perform an endovascular catheterization of a patient to access a shunt already in place in the left ventricle of the patient's heart will be described in detail.

A preferred embodiment of the current invention involves a method of passing a radio-opaque chemical contrast fluid through a shunt which has been installed in the wall of a patient's heart for the purpose of allowing oxygenated blood to flow from within a chamber of the heart directly into a coronary artery. A common reason for performing such a task is to enable imaging of the heart and the blood flow in the arteries surrounding the heart to determine the efficacy of the shunt in providing improved flow in the coronary artery.

To begin such a catheterization procedure, the distal end of the catheter 14 is inserted into the femoral artery 10 of a patient, via a site 12 in the patient's groin. The distal end of catheter 14 (shown in FIG. 8) is then advanced along femoral artery 10 in retrograde fashion. Upon reaching the upper most extension of the femoral artery, catheter 14 is then directed into the descending aorta 16. From descending aorta 16, catheter 14 is further advanced in retrograde fashion into the arch of aorta 18. Advancing through arch of aorta 18 retrograde, the distal end of catheter 14 passes through the ascending aorta 20 directly into the heart 26. Preferably, catheter 14 is advanced into a heart chamber 22 through the aortic valve 24. In FIGS. 7 and 8, the catheterization has been to the left ventricle of a patient's heart. First segment 145 is preferably of sufficient length to permit the insertion of catheter 14 in femoral artery 10 of a patient and extension of catheter 14 into patient's heart 26.

In FIG. 8, an enlarged view of the left ventricle of the patient is shown, with the catheter 14 entering chamber 22 from ascending aorta 20 and a shunt 30 in place in the wall 32 of heart chamber 22 being shown. Note that the basket 102 at distal end 104 of the inner tube 106 has remained in a collapsed position within the outer sheath 108 as catheter 14 was inserted into heart chamber 22. Catheter 14 is advanced into heart 26 so that first segment 145 extends through aortic valve 24. Primary curve 146 rests substantially on the inferior wall of heart chamber 22 with second segment 147 extending superior within the chamber 22. Secondary curve 148 directs third segment 149 substantially anterior.

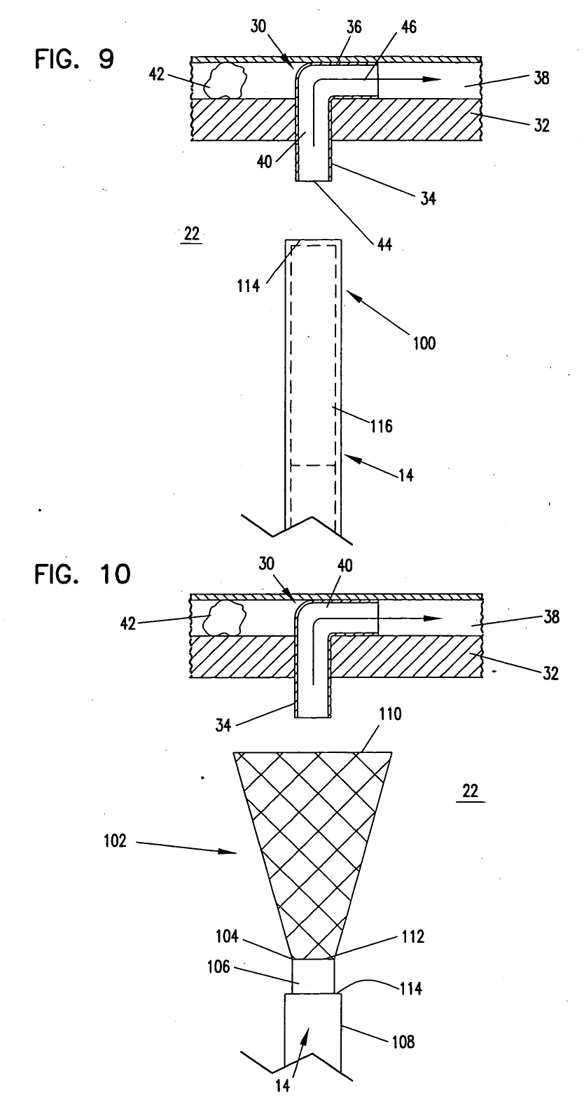

Shunt 30 is located on the anterior wall of chamber 22 and includes two ends, the first end 34 (shown in FIG. 9) extending into heart chamber 22 through heart wall 32, and the second end 36 (shown in FIG. 9) extending into a coronary artery 38 (illustrated is the left anterior descending coronary artery). It is anticipated that second end 36 of shunt 30 may be placed in any of the coronary arteries extending across the left ventricle. First end 34 and second end 36 have openings 44 and 46, respectively, which are connected by an open passageway 40 through the center of the shunt. The first end of the shunt extends into the heart chamber beyond wall 32 of the heart. This protrusion of first end 34 facilitates the stabilization of the diagnostic catheter 14. Opening 46 in second end 36 is directed so that blood flowing through shunt 30 from heart 26 will exit opening 46 in the direction of normal blood flow in coronary artery 38, the direction of normal blood flow being shown by the arrow in FIGS. 9 through 14. Also in FIGS. 9 through 14, an occlusion 42 is shown in coronary artery 38 upstream from shunt 30. It is anticipated that catheters conforming with the present invention may be used with other stent configurations as well (e.g., valved, unvalved, natural graft, mesh, flexible rigid, etc.) Also, catheters conforming with the present invention could be used to access side anastomosis sites.

Once third segment 149, secondary curve 148, second segment 147, primary curve 146 and a portion of first segment 145 of catheter 14 has entered heart chamber 22 via ascending aorta 20, distal end 100 can be directed to the vicinity of first end 34 of shunt 30 in heart wall 32, as shown in FIG. 9. The relative orientation of primary and secondary curves 146 and 148 and the angular offset of third segment 149 allow the distal end of catheter 14 to be directed to any of the interior of chamber 22. Preferably, third segment 149 is coaxially aligned with first end 34.

When in position near first end 34 of shunt 30, distal end 112 of outer sheath 108 is retracted with respect to distal end 104 of inner tube 106 to uncover collapsed basket 116 attached to distal end 104 of inner tube 106, thus permitting collapsed basket 116 to expand to expanded basket 102, as shown in FIG. 10.

Expanded basket 102 includes a wide end 110 which is cone shaped and located opposite of a narrow end 112, narrow end 112 being attached to distal end 104 of inner tube 106. Expanded basket 102 is of an open design so that wide end 110 and narrow end 112 are in fluid and physical communication with each other. Once expanded basket 102 has been allowed to expand, expanded basket 102 is positioned so that wide end 110 of expanded basket 102 overlays upon first end 34 of shunt 34 in heart wall 32, as shown in FIG. 11.

After expanded basket 102 has been overlaid on first end 34 of shunt 30, distal end 114 of outer sheath 108 of catheter 14 is advanced with respect to distal end 104 of inner tube 106, so that distal end 114 of outer sheath 108 once again begins to interfere with expanded basket 102 and cause basket 102 to collapse, reverting back to collapsed basket 116. As expanded basket 102 collapses to become collapsed basket 116, wide end 110 is narrowed until it contacts first end 34 of shunt 30 and captively holds distal end 100 of catheter 14 to shunt 30, as shown in FIG. 12.

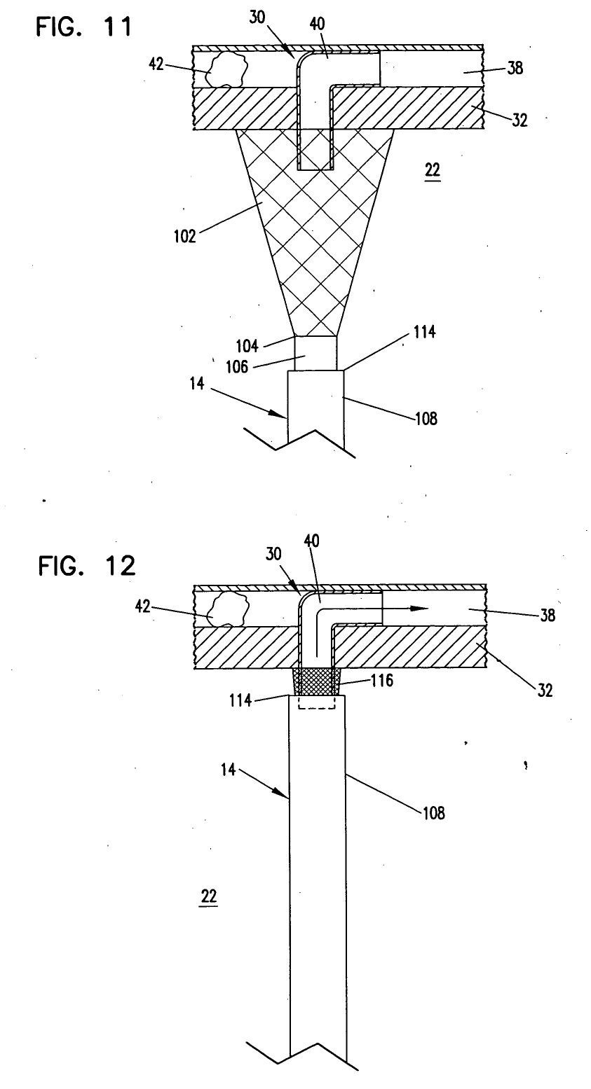

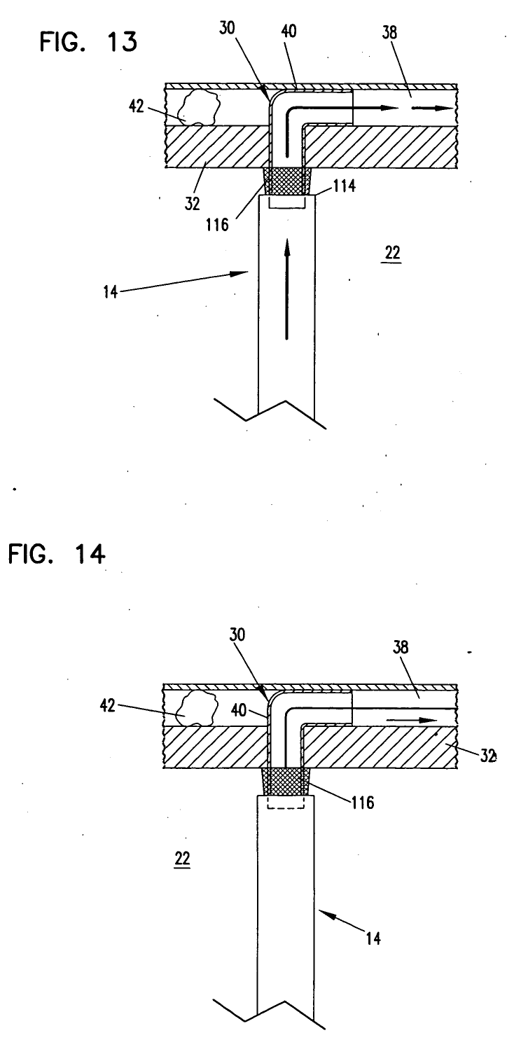

With catheter 14 now stabilized with respect to any movement of shunt 30 caused by movement of heart wall 32 due to normal contractions of heart 26, a radio-opaque fluid 120 can be passed through inner tube 106 of catheter 14 and flow straight through the distal end of catheter 14, into shunt 30 and into coronary artery 38, as shown in FIG. 13.

Alternatively, another embodiment of the method of the invention is shown in FIG. 14. In this embodiment, the steps are identical to the steps above, except, a wire 130 is passed though inner tube 106 and through shunt 30 into coronary artery 38 instead of radio-opaque fluid 120. Wire 130 can then be used as the foundation for performing a variety of other procedures within coronary artery 38 downstream of shunt 30. These procedures might include but not be limited to, inserting an arterial stent in the coronary artery, or performing angioplasty, atherectomy or pyroplasty in the coronary artery.

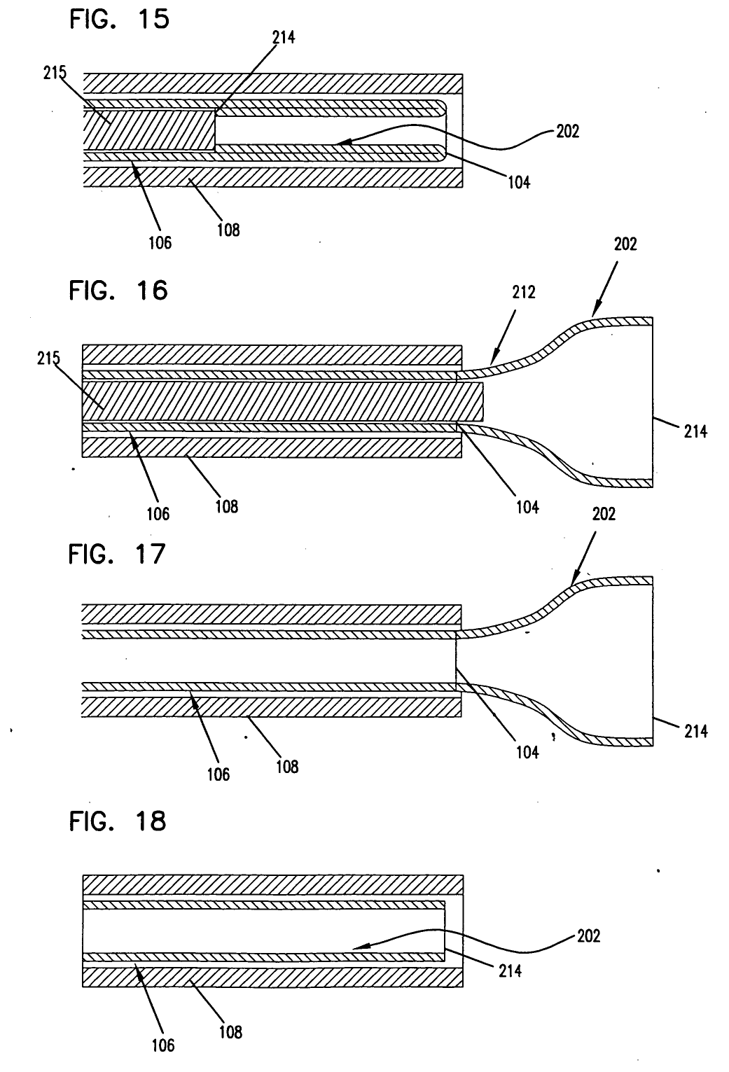

Further alternative embodiments for distal end 104 of inner tube 106 are shown in FIGS. 15 through 21. FIGS. 15 through 18 illustrate a trumpet 202, which operates in a similar fashion to basket 102. Trumpet 202 includes a narrow end 212 and a wide end 214, with narrow end 212 attached to distal end 104 of inner tube 106. FIG. 15 shows trumpet 202 configured for insertion into a patient, with wide end 214 inverted within the hollow interior of inner tube 106. Once the catheter 14 is positioned within heart chamber 22, a shaft 215 is extended through the interior of inner tube 106 to eject wide end 214 and allow trumpet 202 to expand, as shown in FIG. 16. Shaft 215 is then withdrawn from catheter shaft 156, as shown in FIG. 17 allowing trumpet 202 to be used in the same fashion as described above with regard to expanded basket 102 to capture end 34 of shunt 30. For withdrawal from heart chamber 22, wide end 214 is retracted within distal end 100 of outer sheath 108, as shown in FIG. 18.

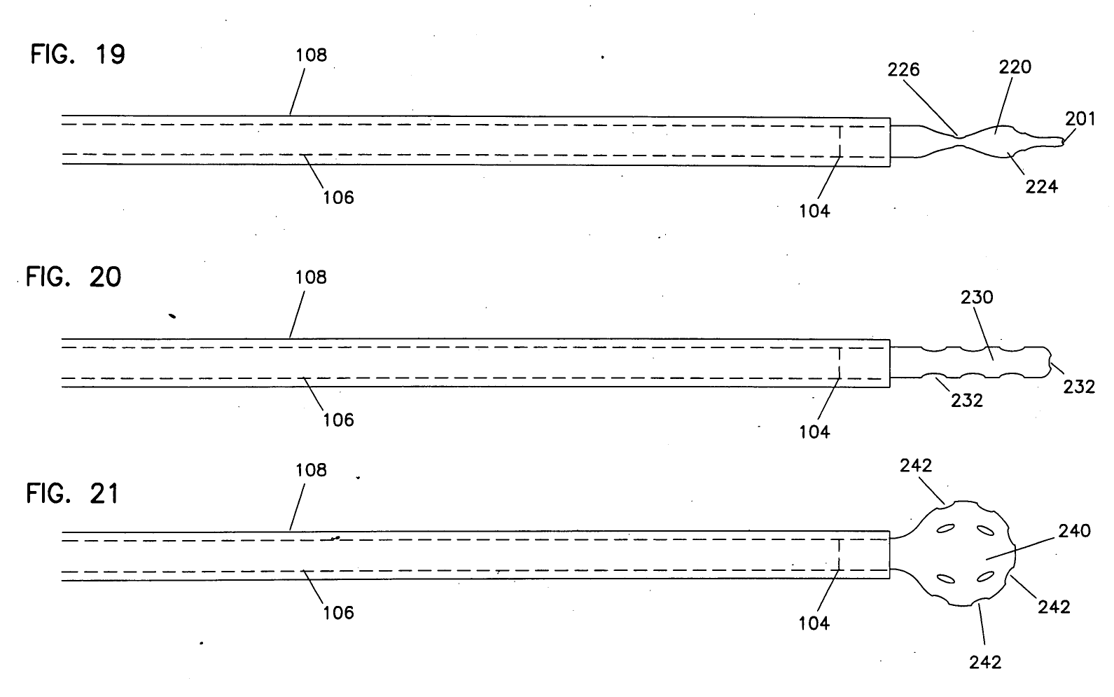

FIGS. 19 through 21 show alternative embodiments of devices that may be attached at distal end 104 of inner tube 106 for injecting dye into heart chamber 22. FIG. 19 shows a bullet or torpedo shaped inner catheter distal end device 220 with a tapered or narrowed waist 226 attached at distal end 104 of inner tube 106. At the extreme distal end of device 220 is an opening 222 and along device 220 extending radially beyond outer sheath 108 are a series of smaller openings 224. The openings 222 and 224 allow fluid to be injected to heart chamber 22 through inner tube 106. Inner catheter distal end device 230, shown in FIG. 20, provides an end to inner tube 106 cylindrically shaped with a series of similarly sized openings 232 along the sides and at the extreme distal end of the device. Device 230 is attached to distal end 104 of inner tube 106 and permits fluid to be injected through catheter 14 into heart chamber 22. FIG. 21 shows a balloon shaped inner catheter distal end device 240 attached to distal end 104 of inner tube 106. Device 240 incorporates a series of spaced-apart openings 242 which permit fluid to be injected through catheter 14 into heart chamber 22. Device 240 is held collapsed within outer sheath 108 until outer sheath 108 has entered heart chamber 22. Inner tube 106 is then extended relative to outer sheath 108 as shown in FIG. 21, allowing device 240 to expand into a balloon shape.

Having described preferred aspects and embodiments of the present invention, modifications and equivalents of the disclosed concepts may readily occur to one skilled in the art. However, it is intended that such modifications and equivalents be included within the scope of the claims which follow.

1-42. (Cancelled).

43. A method for treating a heart, comprising:
inserting a catheter into the heart;
placing the catheter in flow communication with a device previously implanted in a heart wall; and
performing at least one of a diagnostic operation and a therapeutic operation on the heart via the flow communication.

44. The method of claim 43, wherein the catheter is inserted into a heart chamber.

45. The method of claim 44, wherein the heart chamber is the left ventricle.

46. The method of claim 44, wherein the catheter is inserted into the heart chamber via a heart valve.

47. The method of claim 44, wherein the catheter is configured to allow a distal end of the catheter to be directed to a desired portion of the heart chamber.

48. The method of claim 43, wherein the catheter includes at least one curve to assist in placing the catheter in flow communication with the device.

49. The method of claim 48, wherein the at least one curve is preformed.

50. The method of claim 48, wherein the at least one curve includes two curves.

51. The method of claim 48, wherein the at least one curve corresponds to an anatomic shape of a heart chamber.

52. The method of claim 43, wherein the catheter includes an inner tube and an outer sheath disposed around the inner tube and movable relative to the inner tube.

53. The method of claim 43, wherein the catheter includes an expandable distal member.

54. The method of claim 53, wherein the placing step includes contracting the expandable member to capture an end of the device.

55. The method of claim 53, wherein the placing step includes expanding the expandable distal member, positioning the expanded distal member around an end of the device, and contracting the expandable distal member to capture the end of the device.

56. The method of claim 53, wherein the expandable distal member is inserted into the heart in a contracted configuration.

57. The method of claim 43, wherein the catheter includes a distal member that tapers from a distal end to a proximal end.

58. The method of claim 43, wherein the placing step includes capturing an end of the device.

59. The method of claim 43, wherein the placing step includes collapsing a distal member of the catheter around an end of the device.

60. The method of claim 59, wherein the end of the device extends into a heart chamber.

61. The method of claim 43, wherein the placing step includes placing a distal end of the catheter adjacent to an end of the device.

62. The method of claim 43, wherein an end of the device extends into a heart chamber.

63. The method of claim 43, wherein the device extends through the heart wall.

64. The method of claim 43, wherein the at least one of the diagnostic operation and the therapeutic operation includes providing a radio-opaque fluid.

65. The method of claim 43, wherein the at least one of the diagnostic operation and the therapeutic operation includes providing a guidewire.

66. The method of claim 43, wherein the at least one of the diagnostic operation and the therapeutic operation includes inserting a stent.

67. The method of claim 43, wherein the at least one of the diagnostic operation and the therapeutic operation includes performing an angioplasty procedure.

68. The method of claim 43, wherein the at least one of the diagnostic operation and the therapeutic operation includes performing an atherectomy procedure.

69. The method of claim 43, wherein the at least one of a diagnostic operation and a therapeutic operation includes performing a pyroplasty procedure.

70. The method of claim 43, wherein the at least one of a diagnostic operation and a therapeutic operation includes imaging.
